(19) United States
(12) Patent Application Publication
(10) Pub. No.: US 2016/0310981 A1
(43) Pub. Date: Oct. 27, 2016

(54) LIQUID DROPLET APPARATUS

(71) Applicant: KONINKLIJKE PHILIPS N.V., Eindhoven (NL)

(72) Inventor: DIRK ERNEST VON HOLLEN, CLARK, NJ (US)

(21) Appl. No.: 15/105,592

(22) PCT Filed: Dec. 16, 2014
(86) PCT No.: PCT/EP2014/078080

§ 371 Date: Jun. 17, 2016

(30) Foreign Application Priority Data
Dec. 19, 2013 (EP) 13198579.8

(51) Int. Cl. B05B 17/06; A61M 16/00; A61M 11/00; B05B 17/00

(52) U.S. Cl. CPC B05B 17/0653; B05B 17/0646; A61M 16/0054; A61M 11/005

(57) ABSTRACT

Apparatus for producing liquid droplets (for example an aerosol) is described. The apparatus comprises a vibratory element (106) arranged, in use of the apparatus, to vibrate an aperture plate (102) having at least one aperture therein. Also provided is a sealing element (202), which is arranged to provide a liquid tight seal over at least a portion of the vibratory element (106). The sealing element (202) comprises a closed cell foam material. In some embodiments, sealing element (202) may be molded over other elements of the apparatus, including the vibratory element (106).

### FIELD OF THE INVENTION

The invention concerns apparatus for producing liquid droplets, for example for use in ultrasonic or vibrating mesh aerosol nebuliser devices.

### BACKGROUND OF THE INVENTION

Liquid droplet generators such as aerosol generators are used in various industries to produce droplets. For example, nebulisers deliver pharmaceuticals in droplet form for inhalation by a patient.

There are many known nebuliser designs, which include human and spring powered devices. Much recent research has been directed to the use of electrically powered nebulisers, for example jet nebulisers (also called atomizers), which force a gas through a liquid containing medicine; ultrasonic wave nebulisers, in which a piezoelectric element vibrates a column of liquid to produce a vapour mist; and vibrating mesh technology, in which an aperture plate (which may be mesh, a membrane, a machined metal plate or the like) is vibrated against the surface of a liquid reservoir, or has a supply of liquid applied directly to the aperture plate. When the aperture plate is vibrated (usually by a piezoelectric element), droplets are dispensed.

A particular example of a nebuliser using vibrating mesh technology is a ring style mesh aerosol system. An example of aerosol generating apparatus 100 of such a system is shown schematically in FIGS. 1A and 1B. As can be seen from the exploded view of FIG. 1 and the cross sectional view of FIG. 1B, in this example, a mesh 102 is gravity fed and is vibrated within a housing 110 to dispense droplets. The mesh 102 is mounted on a washer 104 which is attached to a piezoelectric element 106 via an adhesive layer 108. The mesh 102, piezoelectric element 106, washer 104 and adhesive layer 108 are all arranged within the housing 110. As illustrated in the Figures, a liquid is arranged above the mesh 102 and, on operation of a nebuliser containing the arrangement, an aerosol mist is dispensed below the apparatus 100. Such apparatus 100 may be part of a liquid nebuliser drug delivery system or a humidification system for ventilation apparatus, or attached to a nasal cannula, or the like.

In order to prevent fluid from contacting the piezoelectric element 106 or electrical contacts in a ring style aerosol generator, it is known to provide a seal, for example a silicone injection overmolding, or o-rings or gaskets in contact with rear of the mesh 102. One process for overmolding such an apparatus is described in US2004/0050947, assigned to Aerogen, Inc, in which a sealing member formed of elastomers, rubbers or the like is described. Over time, such seals undergo vibration and are exposed to various compositions (drug solutions and suspensions, cleaning liquids and the like), which can degrade their integrity. Once leaks occur, this can result in nebuliser failure. There is a desire to ensure that the piezoelectric element 106 remains isolated from any liquid. Therefore, there is a desire to ensure that such seals remain intact.

In an ultrasonic wave nebuliser, for example as described in WO2004/002558, it is known to provide a protective layer such as an aluminium oxide layer, which is applied (for example using adhesive) to the surface of a piezoelectric element over an area which may otherwise contact a liquid. In WO2004/002558, a compressed seal is arranged about the periphery of such a protective layer.

### SUMMARY OF THE INVENTION

According to a first aspect of the invention, there is provided apparatus for generating liquid droplets, the apparatus comprising a vibratory element arranged, in use of the apparatus, to vibrate an aperture plate; and a sealing element arranged to provide a liquid tight seal over at least a portion of the vibratory element, wherein the sealing element comprises a closed cell foam material.

Closed cell foam is a substance that is formed by trapping discrete pockets of gas in an encapsulating material such as elastomer, rubber, silicone, plastics, cellular polymeric material or the like. As the pockets of gas are mostly separate, the material can act as a fluid barrier. In contrast to other, more solid or less compressible substances (for example solid silicone), such material can provide a fluid barrier without significantly impeding the operation of a vibratory element (such as a piezoelectric element). The sealing element may therefore be arranged to provide a liquid tight seal without substantially damping the motion of the vibratory element. In other words, the compression modulus of closed cell foam can often be low when compared to other materials capable of forming a resilient seal. This in turn means a seal may be provided without requiring a significantly higher power input to create the same oscillation of the vibratory element absent a seal. It may also be relatively low density, thus not adding weight to the apparatus unduly, which may be desirable in particular if the apparatus is intended to be portable (as may be the case, for example, for some nebuliser and/or ventilation apparatus). In addition, the geometry of the cells can be varied to achieve different foam properties, which may include its compressibility. Materials which are relatively unreactive (i.e. chemically stable) may be used to ensure the longevity of the seal, such that it retains its integrity over time.

The sealing element may be arranged around at least a portion of the vibratory element. Where the apparatus is a ring style mesh aerosol system, the sealing element may be annular, for example a continuous and complete annulus, although the skilled person will be aware that other forms may also be suitable, depending at least in part on the shape and configuration of the vibratory element.

An aperture plate having at least one aperture therein and a first and second side may be provided as part of the aerosol apparatus, the arrangement being such that, in use, liquid is supplied to the first side of the aperture plate, and the vibratory element vibrates the aperture plate to cause the liquid to be dispensed from the second side of the aperture plate as an aerosol or droplets. Such an arrangement will be familiar to a skilled person from known aerosol apparatus.

As will be appreciated by the skilled person, driving the vibratory element of such an apparatus (which may be referred to as a ‘vibrating mesh’ style apparatus) is generally a relatively low power operation when compared in particular to driving a vibratory element in related apparatus such as ultrasonic wave nebulisers. As will be familiar to the skilled person, an ultrasonic wave nebuliser generally contains a piezoelectric element that is typically mounted on a seal such as an O-ring and surrounded by a protective layer. Often, this is a metallic material such as stainless steel, brass or an alloy. This protective layer of material protects the piezoelectric element from damage from fluid but allows vibrations to be transmitted through the protective layer into the fluid, and then transmitted through the fluid until it reaches the surface. The fluid then breaks up into droplets into the air. The use of the protective layer impedes the transmission of the vibration, which in turn requires for more power to be required to nebulise fluid (i.e. turn it into droplets) compared to a vibrating aperture plate technology.

The lower energy of a vibrating aperture plate nebuliser is in many ways desirable as it means that the energy requirements of such apparatus are low. However, it also means that the vibratory apparatus, being low powered, is more likely to suffer from damping than, for example a vibratory element in ultrasonic wave nebuliser, which will be driven at higher powers. Therefore, providing a seal which does not excessively damp its motion is preferable.

The sealing element may be in contact with, or directly bonded to, such an aperture plate. This provides for an effective seal. It will be appreciated that the function of an apparatus including an aperture plate requires that the aperture plate be able to vibrate relatively freely. Through use of a sealing element as described herein, such a seal may be provided without excessive damping.

In particular examples, the sealing element may be an overmolded element. This is a convenient way of providing a fluid tight seal. For example, it may be an overmolded element which can be produced using injection molding processes. However, as such overmolding is usually carried out at a relatively high temperature, such techniques (like injection molding) may risk damage to heat sensitive parts of the apparatus, such as the vibratory element. Therefore, in other examples, the sealing element may be formed separately (for example, die cut) and arranged about the vibratory apparatus.

Although in some examples the sealing element may be held in place for example using an interference fit, in other examples, adhesive is provided between at least a portion of the apparatus and the sealing element (for example, adhesive may be provided between the sealing element and the aperture plate, and/or between the sealing element and any contacting component, including any housing). In some embodiments, the sealing element may be at least partially transparent and the adhesive is an ultraviolet cured adhesive. This allows it to be cured after the sealing element is in place (although other adhesives could be used as will be familiar to the skilled person).

Alternatively or additionally, there may be a chemical bond between sealing element and at least a portion of an element of the apparatus over which it is formed or arranged, even in the absence of an adhesive. This may be achieved through selection of the materials for these elements. There are various known substances which, in combination, form such a bond, as will be familiar to the skilled person. In particular, chemical agents may be included in a foam material to enhance its bonding to surfaces (and therefore enhance its ability to provide a fluid tight seal).

Preferably, the apparatus is arranged such that, absent a vibration applied by the vibratory element, the sealing element is substantially uncompressed. This is advantageous as the full benefit of its compressibility will be available when the vibratory element acts on the sealing element: in contrast, if the seal is already partially compressed before the vibratory element is operated, its effective compressibility is lower and the sealing element may impart comparatively more damping to the action of the vibratory element.

According to a second aspect of the invention, an aerosol apparatus comprises a vibratory element arranged, in use of the apparatus, to vibrate an aperture plate; and a sealing element arranged to provide a liquid tight seal over at least a portion of the vibratory element, wherein the sealing element comprises a closed cell foam material.

The apparatus may have any of the features discussed in relation to the first aspect of the invention.

According to a third aspect of the invention, there is provided a method of manufacture of an apparatus for producing liquid droplets comprising:
i. providing a vibratory element arranged, in use of the apparatus, to vibrate an aperture plate;
ii. arranging a sealing element over at least a portion of the vibratory element to provide a liquid tight seal over at least a portion of the vibratory element, wherein the sealing element comprises a closed cell foam material.

Before the step of arranging a sealing element about the vibratory element, an adhesive may be provided, and arranged to hold the sealing element in place. If desired, such an adhesive could be cured after the step of arranging a sealing element about the vibratory element.

The step of arranging may comprise overmolding, which may in turn comprise injection molding. The step of arranging may comprise arranging the sealing element around at least a portion of the vibratory element.

The method may also comprise selecting the material of the sealing element such that, when in situ, the sealing element will not significantly impede the vibrations of the vibratory element.

The method may comprise a method of manufacture of an aerosol apparatus according to the first aspect, or of apparatus for producing droplets according to the second aspect.

According to a fourth aspect of the invention, there is provided a sealing element for an apparatus for producing liquid droplets, the sealing element comprising a closed cell foam material, wherein the sealing element is arranged to provide a liquid tight seal over at least a portion of a vibratory element of an apparatus for producing liquid droplets. The sealing element may be such that it does not significantly impede the vibrations of the vibratory element. The sealing element may be a sealing element as described in relation to any of the first, second or third aspects of the invention.

The apparatus according to the first or second aspect of the invention may be arranged for use in a nebuliser and/or ventilation apparatus.

Embodiments of the in the invention are now described, by way of example only, with reference to the following Figures.

### BRIEF DESCRIPTION OF THE DRAWINGS

FIGS. 1A and 1B show a known configuration of an aerosol generating apparatus for use in a nebuliser; and

FIGS. 2A-2D show embodiments of aerosol generating apparatus.

### DETAILED DESCRIPTION OF EMBODIMENTS

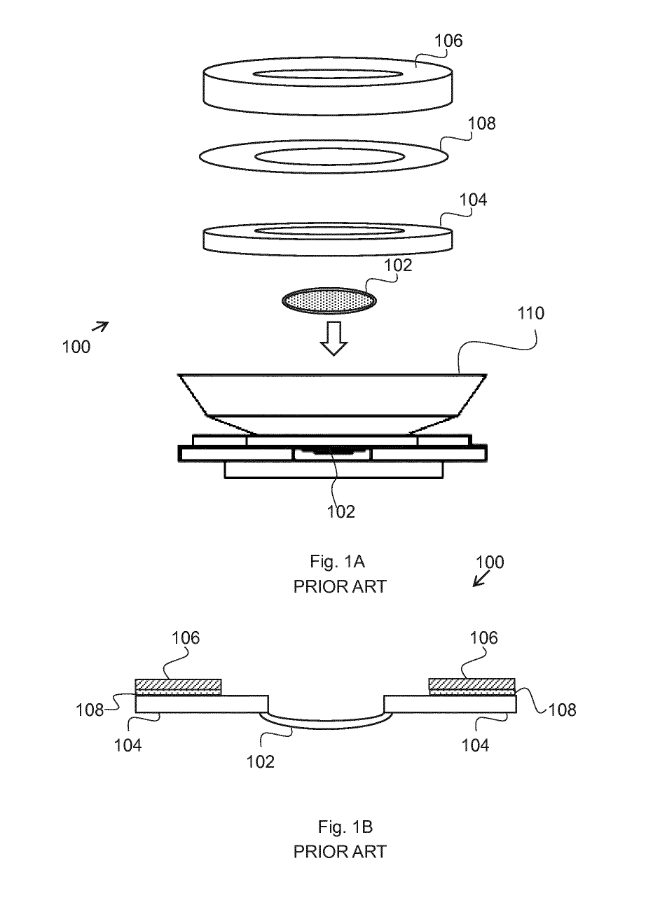

As noted above, FIG. 1A shows an exploded view of a known configuration of a vibrating mesh apparatus 100 for use in a nebuliser in which an aperture plate, provided in this example by a mesh 102, is vibrated by a vibratory element, which in this example is an annular piezoelectric element 106, against a surface of a liquid reservoir within a housing 110 to dispense droplets. FIG. 1B shows a cross sectional view of the components, from which it can be seen that the mesh 102 is mounted on a platform provided by a washer 104, which is in turn mounted on the piezoelectric element 106 and in this example attached thereto by an adhesive layer 108.

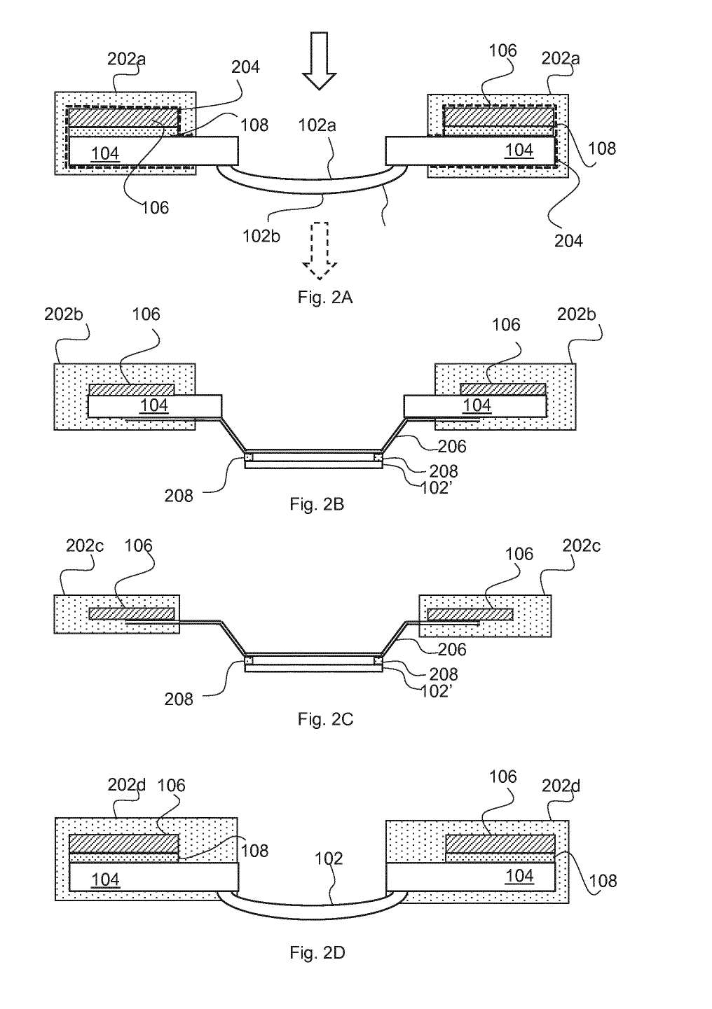

FIGS. 2A-2C show configurations of apparatus for producing liquid droplets, which in this example are aerosol apparatus and incorporate a sealing element 202, which may be an overmolding. As shown in FIG. 2A (but is also true of other aperture plates mentioned herein), the mesh 102 has a first side 102a, to which liquid may be supplied (as indicated by the solid line arrow) and a second side 102b, from which droplets may be dispensed (as indicated by the broken line arrow). Throughout the drawings, like parts are indicated with like reference numerals. While the embodiments of FIGS. 2A to 2C are in a ‘gravity fed’ orientation, the skilled person will appreciate that such ‘vibrating mesh’ technology can be gravity fed or arranged such that the supply of liquid is below the mesh 102. The teaching as set out herein could be readily applied to either configuration, and to variations thereof.

The skilled person will appreciate that the function of the sealing element 202 could be provided by a sealing element which is formed, then placed around the components. Such a sealing element may be, for example, die cut, or otherwise formed to have a complementary shape to the underlying components. Indeed, in some examples, this may be preferred to overmolding, as overmolding often requires relatively high temperatures and some apparatus (such as some piezoelectric elements) can be damaged from exposure to such temperatures.

FIG. 2A shows a similar configuration to that shown in FIG. 1B. However, in FIG. 2A, a portion of the apparatus is provided with an annular sealing element 202a comprising a closed cell material. In this example, the mesh 102 is a domed mesh. It will be noted that the sealing element 202a is arranged over the piezoelectric element 106 and extends to the underside of the washer 104, although this need not be the case in all embodiments. In this example, the sealing element 202a fully encapsulates the piezoelectric element 106. However, this need not be the case in all examples. In some examples, just a portion of the piezoelectric element 106 may be overmolded, for example the portion comprising electrical connections (not shown).

In this embodiment, it will be noted that the sealing element 202a comprises a portion which is arranged over the face of the piezoelectric element 106, i.e. is substantially perpendicular to the direction of movement during vibration. In other words, the sealing element 202a is arranged such that it will be acted upon during vibration, and specifically will undergo repeated compressions during the operation of the piezoelectric element 106.

Using a closed cell material to provide the sealing element 202a in place of solid (or less compressive) materials such as solid silicone allows the piezoelectric element 106 to vibrate without excessive damping. Closed cell foam encapsulation, which may comprise, for example an elastomer, rubber or silicone material formed in a closed cell structure provides a sealing feature to isolate the vibratory element with minimal restriction to its operation. By arranging the sealing element 202a about the piezoelectric element 106 (for example as an overmolding), a good seal may be formed around the piezoelectric element 106.

The skilled person will be aware of various foam constructions, which may be referred to in various ways. For example, closed cell silicone sponge, which may be referred to simply as silicone sponge or expanded silicone, usually refers to a substance derived from a sheet of silicone gum material which expands on curing to form closed cells. It is usually possible to control the thickness of the sponge. As mentioned above, the term ‘closed cell’ means that, in general, the cells which form are generally non-interconnected and thus liquids are prevented from travelling (for example by a wicking action) through the material. This can be contrasted with so called silicone ‘foam’, which term generally refers to a comparatively light weight material with an at least partially open cell structure. A silicone foam is usually made of liquid silicone cured in an oven. Generally, silicone foam, being an open cell structure, only functions as a liquid seal if held in compression.

As the sealing element 202a is intended to allow vibration, it may be preferred to use a sealing material which is generally, absent a vibration, substantially uncompressed (as in the examples provided herein). This in turn allows for compression when the piezoelectric element 106 is operated.

In addition, it will be appreciated that the sealing element 202a could be connected to a housing, for example a housing 110 substantially as shown in FIG. 1. It could be attached thereto with an adhesive (e.g. an epoxy) or otherwise mounted securely (for example between two retaining flanges, within a recess, or the like). It may be secured around its periphery only, as securing the assembly via the outer perimeter provides the least amount of resistance to the vibration.

In addition, while in the illustrated embodiment, the sealing element 202a does not extend to the mesh 102, it could in other examples extend to cover at least a portion (for example, the perimeter) of the mesh 102.

It will also be appreciated that, when the skilled person refers to a ‘closed cell’ material, this refers to materials in which a relatively high proportion of cells are discrete, or the proportion of interconnected cells is relatively low.

Forming an overmolding around an object may comprise providing an overmolding substance in a fluid form and allowing it to solidify around the object. Such a process can result in a mechanical bond being formed, as, once the overmolding solidifies, the object which has been overmolded may comprise features, such as protruding regions, which prevent the overmolding from being readily removed. However, in order to improve the integrity of the overmolded apparatus and/or the sealing properties of the sealing element 202a, in this example, an adhesive 204 is applied to the structure before the sealing element 202a is in turn applied, then allowed or caused to cure. In particular, in this example, the sealing element 202a is transparent, and the adhesive 204 is a UV cured adhesive. This can be cured through the sealing element 202a once it is in place using UV light.

FIG. 2B shows an alternative embodiment. In this example, a mesh 102′ is not mounted in the washer 104, but is suspended from a flexible platform 206 to which it is attached with adhesive 208. The flexible platform 206 transfers vibrations from the piezoelectric element 106 to the mesh 102′ (which in this example is a flat screen mesh).

Again, in this embodiment, a sealing element 202b is arranged to ensure that the piezoelectric element 106 is isolated from any liquid which is conveyed to the mesh 102′. In this example, the sealing element 202b again extends across the whole piezoelectric element 106, fully encapsulating it so that it is sealed on all sides, although, as noted above, this need not be the case.

The sealing element 202b therefore provides an annular ‘cushion’ to support the vibrating elements structure, while still allowing the piezoelectric element 106 to perform its function with minimal damping.

In the example of FIG. 2B, the sealing element 202b includes properties to provide a chemical as well as a mechanical bond to the support surface which improves the lifespan of its sealing capability. Therefore, as the sealing element 202b holds the piezoelectric element 106 to the washer 104, the adhesive layer 108 has been omitted. However, in some examples, the adhesive layer 108 may be omitted even in the absence of such chemical bonding between a sealing element and other parts of the apparatus, as the mechanical bond formed between the components (for example in overmolding) may be sufficient to secure elements of the apparatus together.

FIG. 2C shows an embodiment which is similar to that shown in FIG. 2B, but in this example, it will also be noted that the washer 104 has been removed and the piezoelectric element 106 is mounted directly on the flexible platform 206. The sealing element 202c extends to the underside of the flexible platform 206, although this need not be the case in all embodiments.

FIG. 2D shows an embodiment which is similar to that shown in FIG. 2A, but in this example, the adhesive 204 has been omitted (although may be included in other embodiments) and a die cut closed cell sealing element 202d is provided which extends to contact the perimeter of a domed mesh 102. Providing a sealing element 202d which extends to the perimeter of the mesh effectively seals the piezoelectric element 106. As will be familiar to the skilled person, in some examples, the outer perimeter of a vibrating aperture plate may be free of apertures and therefore can be covered with a sealing element 202d without sealing off apertures.

While the invention has been illustrated and described in detail in the drawings and foregoing description, such illustration and description are to be considered illustrative or exemplary and not restrictive; the invention is not limited to the disclosed embodiments. Features from one embodiment, or described as variants of a particular embodiment, may be combined or replaced with features from another embodiment.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. Any reference signs in the claims should not be construed as limiting the scope.

1. Apparatus for generating liquid droplets comprising
a vibratory element arranged, in use of the apparatus, to vibrate an aperture plate;
a sealing element arranged to provide a liquid tight seal over at least a portion of the vibratory element, wherein the sealing element comprises a closed cell foam material.

2. Apparatus according to claim 1, comprising an aperture plate having at least one aperture therein, and having a first and second side; and the arrangement being such that, in use, liquid is supplied to the first side of the aperture plate, and the vibratory element vibrates the aperture plate to cause the liquid to be dispensed from the second side of the aperture plate as droplets.

3. Apparatus according to claim 2, in which the sealing element is in contact with, or directly bonded to, the aperture plate.

4. Apparatus according to claim 1, in which the sealing element comprises at least one of the following: an elastomer, rubber, silicone, plastic, cellular polymeric material.

5. Apparatus according to claim 1, in which the sealing element is arranged to provide a liquid tight seal without substantially damping the motion of the vibratory element.

6. Apparatus according to claim 1, in which adhesive is provided between at least a portion of the vibratory element and the sealing element.

7. Apparatus according to claim 6, in which the sealing element is at least partially transparent and the adhesive is an ultraviolet cured adhesive.

8. Apparatus according to claim 1, in which there is a chemical bond between sealing element and at least a portion of an element of the aerosol apparatus.

9. Apparatus according to claim 1, in which the sealing element is an overmolded element.

10. Apparatus according to claim 1, which is an aerosol apparatus.

11. A method of manufacture of an apparatus for generating liquid droplets comprising:
providing a vibratory element arranged, in use of the apparatus, to vibrate an aperture plate; and
arranging a sealing element to provide a liquid tight seal over at least a portion of the vibratory element, wherein the sealing element comprises a closed cell foam material.

12. A method according to claim 11, which further comprises, before the step of arranging a sealing element, providing an adhesive arranged to hold the sealing element in place.

13. A method according to claim 12 which further comprises, after the step of arranging the sealing element, curing the adhesive.

14. A method according to claim 11, which further comprises selecting the material of the sealing element such that, when in situ, the sealing element will not significantly impede the vibrations of the vibratory element.

15. (canceled)
